(19) United States
(12) Patent Application Publication
(10) Pub. No.: US 2010/0227338 A1
(43) Pub. Date: Sep. 9, 2010

(54) Method and Apparatus for Rapid Detection and Identification of Live Microorganisms Immobilized On Permeable Membrane by Antibodies

(75) Inventor: Sergey Gazenko, Mason, OH (US)

Correspondence Address: THOMPSON HINE L.L.P.;Intellectual Property Group, P.O. BOX 8801, DAYTON, OH, 45401-8801, US

(73) Assignee: NANOLOGIX, INC., Hubbard, OH (US)

(21) Appl. No.: 12/785,180

(22) Filed: May 21, 2010

Related U.S. Application Data
(63) Continuation-in-part of application No. 12/532,501, filed on Mar. 16, 2010.
(60) Provisional application No. 60/896,321, filed on Mar. 22, 2007.

(51) Int. Cl. G01N 33/569

(52) U.S. Cl. 435/7.31; 435/7.2; 435/7.32; 435/287.2

(57) ABSTRACT

An apparatus and method is provided for the rapid detection, identification and/or enumeration of one or more live target microorganisms growing on solid nutrient media or filtration membrane. Microcolonies or colonies attach to a porous transfer permeable membrane loaded with immobilized antibodies specific for target microorganisms. Non-specific cells are washed out of the permeable membrane and the membrane then is placed on a container containing a chromogenic and/or a fluorogenic substrate and incubated to allow coloration and subsequent identification and enumeration of color spots of targeted microorganisms and patterns of microcolonies or colonies.

### CROSS REFERENCE TO RELATED APPLICATIONS

This application is a continuation-in-part of U.S. application Ser. No.12/532,501, filed Mar. 16, 2010, which is a Section 371 of PCT/US08/003826, filed Mar. 24, 2008, which claims priority to U.S. Provisional Application No. 60/896,321, filed Mar. 22, 2007, all of which are incorporated herein by reference.

### BACKGROUND OF THE INVENTION

1. Field of the Invention

The present invention relates generally to the field of microbiology and, in particular, to microbiological diagnostics for the rapid detection, identification and/or enumeration of colonies or microcolonies of live microorganisms.

2. Description of the Prior Art

Modern microbiological analysis is based on two main trends: 1) analysis without preliminary growth and 2) analysis after a preliminary growth. The first trend of analyzing without preliminary growth includes methods such as: i) immunological analyses [e.g., immunofluorescence, radioimmunoassay, enzyme immunoassay (EIA) for single cells, etc.]; ii) DNA/RNA analyses via polymerase chain reaction (PCR); and iii) flow cytometry (FC) analyses (detection of single cells after labeling with fluorescent antibodies or fluorogenic substrates). Artificial substrates are also used for detection and analysis of cells by microscopic means. Nevertheless, some of these methods (PCR and immunology) do not distinguish between living and dead cells, which may be important depending upon the test desired. For example, the detection and identification of live cells in different medical, biotechnological, food, agricultural, pharmaceutical, environmental, and military samples is still very important for many human needs.

The frequently used tests that do detect live cells in microbiology [chromatography of fatty acids, enzyme-linked immunosorbent assay (ELISA), mass-spectrometry, Fourier transform infrared (FTIR) spectroscopy, immuno-analyses, etc.] require preliminary growth of microbes, which represents at least one time-consuming initial cell culture before detection and identification can take place. Furthermore some tests like ELISA can be intensive in process steps and chemical reagent materials. While FC in combination with artificial (mainly fluorogenic) substrates is able to detect live cells, FC has drawbacks such as high equipment cost, extensive training requirements, and the need for concentrated of microbiological samples.

Despite these high-tech options, the traditional method of regular colony growth on a Petri plate is still the most common method used to detect live microbes present in a sample. However, analysis by Petri plate can be time and labor intensive. A typical analysis begins with several 10-fold dilutions of a sample followed by the application of one milliliter of the diluted sample distributed evenly over the surface of a nutrient agar. The quantity of 10-fold dilutions can be in any range, e.g., 1 to 12 serial dilutions, with each dilution in a particular range being plated in a Petri plate in order to find a dilution suitable for counting microbial colonies, thereby requiring possibly several to a dozen or more Petri plates. In practice, the correct dilution is found when the number of bacterial colonies on one countable plate does not exceed 250, e.g., as recommended by the USFDA. In order to create colonies, inoculated plates are incubated approximately 24-48 hours for bacteria and 72-120 hours for fungi. Thus a relatively long time is needed to form colonies easily visible to the naked eye. If the sample arises from a time-sensitive biohazard incident, or a hospital patient in critical care, or industrial (food, pharmaceutical) products with short “shelf life” then time is of the essence and time-consuming incubation and serial testing can be a substantial burden with potentially life-threatening or profit lose consequences. Colonies appearing on solid nutrient media are simply counted for detection and enumeration of total microbial growth or are removed and analyzed according to traditional microbiological procedures, e.g., mass-spectrometry, FTIR spectroscopy, chromatography, immunoassays, or PCR.

The removal of a suspicious colony and subsequent analysis of that colony by long and cumbersome traditional methods or complicated and expensive high-tech methods and instruments led to the development of CHROMagar™ nutrient medias. These medias contain special substances, artificial substrates, and antibiotics that allow growth and simultaneous specific coloration of colonies of interest. The CHROMagar™ Candida, CHROMagar™ O157, CHROMagar™ Salmonella, CHROMagar™ Staph aureus and some other are currently used for identification of colonies of interest by pink, green, or blue color of target microorganisms. Unfortunately, the substances initiating coloration tend to collect in the cellular bodies themselves, thereby causing growth problems of the cellular bodies. Therefore, colonies are atypically small and very often need prolonged incubation. Only regular-sized colonies but not microcolonies can be detected because the color is weak, and small light absorption is ineffective for microscopy. This means microcolonies (early stage of colony formation) cannot be detected with the use of CHROMagar™. Only species of interest and some number of other species can grow on CHROMagar™. Therefore CHROMagar™ has only limited usefulness for comprehensive total viable organisms (TVO) microbial detection and enumeration.

Nevertheless rapid, simple and reliable identification of colonies and microcolonies is very important for different areas of medicine, food, biotechnological and pharmaceutical industries, military and civilian defense, and environmental control. For example, the food industry is mainly interested in several food-connected pathogens: e.g., E. coli, Salmonella spp., Listeria spp., Pseudomonas aeruginosa, Staphylococcus aureus, some Lactobacillus spp., Bacillus cereus, some yeast and molds, etc. Reliable monoclonal and polyclonal antibodies exist for a majority of these organisms but they are most useful only after a high quantity of cells are grown on a special media or broth and are identified by EIA or ELISA using sophisticated and expensive equipment like VITEC™ (bioMérieux) by fluorescence or using 96-well plates with specific antibodies immobilized inside the wells.

Consequently, a need arises for a quick, simple, inexpensive and accurate method and apparatus to selectively identify live microorganisms and/or to provide count of live cells that overcomes the limitations of the prior art.

### SUMMARY OF THE INVENTION

The present invention provides an apparatus and methods for the rapid detection, identification and/or enumeration of live microorganisms in a sample after short growth on nutrient media.

In an embodiment, there is provided a method for the rapid detection, identification and/or enumeration of one or more live target microorganisms, such as bacteria, yeast, fungi or eukaryotic cells in a sample. The method comprises placing at least one target microorganism on a nutrient medium to grow at least one microcolony of the at least one target microorganism. After a variable growth period, a permeable membrane is placed on the microcolony(ies). The permeable membrane has immobilized thereon at least one type of antibody specific to an antigen on the target microorganism(s) in the microcolony(ies) in order to bind the target microorganism(s) to the antibody(ies) to form a replica of the microcolony(ies) on the permeable membrane. Any non-bound microorganisms are then washed away. The microcolony(ies) on the permeable membrane is stained to form a stained replica of the microcolony(ies) by placing the permeable membrane on a plate containing, for example, agar, and at least one chromogenic substrate, at least one fluorogenic substrate or a combination of the two dissolved therein to obtain colored and/or fluorescent spots of the target microorganism(s).

In another embodiment, the above-described method further comprises a way to confirm the detection, identification and/or enumeration of the one or more live target microorganisms by placing at least one type of antibody-enzyme conjugate on the permeable membrane. The antibody-enzyme conjugate is specific to the antigen of the at least one target microorganism of the stained replica. The permeable membrane then is incubated for a period of time and any non-bound antibody-enzyme conjugate is washed away from the permeable membrane. A staining solution, such as 3,3′-diaminobenzidine (DAB), is placed on the permeable membrane to obtain colored spots of the target microorganism(s) in the stained replica. Detection, identification and enumeration of the target microorganism(s) from non-target microorganisms is achieved by the difference in the color and intensity of color between the target microorganism(s) in the stained replica and the non-target microorganisms.

In another embodiment, there is provided a method for the rapid detection, identification and/or enumeration of one or more live target microorganisms, such as bacteria, yeast fungi or eukaryotic cells, in a liquid sample. The method comprises placing a permeable membrane having at least one type of immobilized antibody thereon specific to an antigen on or in a liquid sample which may contain at least one live target microorganism so as to form at least one target microorganism antigen-antibody complex. Any non-bound microorganisms are washed away from the permeable membrane. The microorganism antigen-antibody complex(es) is incubated for a period of time to form at least one microcolony on the permeable membrane. The microcolony(ies) on the permeable membrane then is stained by placing the permeable membrane on a plate containing, for example, agar, and at least one chromogenic substrate, at least one fluorogenic substrate or a combination of the two dissolved therein to obtain colored and/or fluorescent spots of the target microorganism(s).

In this embodiment, one or more types of antibodies can be immobilized on the entire surface of the permeable membrane, on a portion of the surface of the permeable membrane, or in portions of the permeable membrane to form signs, shapes or letters. Suitable antibodies for use in the invention include monoclonal antibodies or polyclonal antibodies.

In another embodiment, there is provided a method for rapid detection, identification and/or enumeration of one or more live target microorganisms without the use of immobilized antibodies. The method comprises placing at least one target microorganism on a nutrient medium to grow at least one microcolony of the target microorganism(s). A permeable membrane is placed on the microcolony(ies) to form a replica of the microcolony(ies) on the permeable membrane. The microcolony(ies) then are stained to form a stained replica of the microcolony(ies) by using at least one chromogenic substrate, at least one fluorogenic substrate or a combination of the two to obtain colored and/or fluorescent spots of the at least one target microorganism.

The staining of the microcolony(ies) is performed by placing the permeable membrane on a staining plate containing agar and having the chromogenic substrate(s), the fluorogenic substrate(s)or the combination of the two, dissolved therein. Alternatively, the chromogenic substrate(s), the fluorogenic substrate(s) or a combination of the two can be immobilized directly on the permeable membrane for staining.

In another embodiment, there is provided a device, such as a medical device, for the rapid detection, identification and/or enumeration of one or more live target microorganisms, such as bacteria, yeast, fungi and eukaryotic cells in a sample. The device is comprised of a container having therein nutrient medium to grow at least one microcolony of at least one live target microorganism and a permeable membrane that contacts the microcolony(ies) grown on the nutrient medium. The permeable membrane has at least one type of immobilized antibody thereon specific to an antigen on the target microorganism(s)in the microcolony(ies) in order to bind the target microorganism(s) to the antibody(ies) to form a replica of the microcolony(ies) on the permeable membrane. The device also includes a plate containing, for example, agar, and at least one chromogenic substrate, at least one fluorogenic substrate or a combination of the two dissolved therein to obtain colored and/or fluorescent spots of the target microorganism(s).

In another embodiment, there is provided a test kit system for the rapid detection, identification and/or enumeration of one or more live target microorganisms in a sample. The test kit system comprises a container having therein nutrient medium to grow at least one microcolony of at least one live target microorganism; a permeable membrane that contacts the microcolony(ies) grown on the nutrient medium, in which the permeable membrane has at least one type of immobilized antibody thereon specific to an antigen on the target microorganism(s) in the microcolony(ies) in order to bind the target microorganism(s) to the antibody(ies) to form a replica of the microcolony(ies) on the permeable membrane; a plate containing agar and one or more chromogenic and/or fluorogenic substrates to obtain colored and/or fluorescent spots of the target microorganism(s).

The variable growth period to form microcolonies before detection, identification and enumeration of the at least one target microorganism can be as little as about one-fourth to about one-third less time than usual, in which usual growth time is between about 24 to about 48 hours for bacteria and about 72 to about 120 hours for fungi.

Suitable nutrient media for use in the invention includes, without limitation, agar such as tryptic soy agar.

Suitable chromogenic substrates for use in the invention include, without limitation, tetrazolium dyes such as of MTT (3-(4,5-dimethylthiazol-2-yl)-2, 5-diphenyltetrazolium bromide) and INT (2-(p-iodophenyl-3-(p-nitrophenyl)-5-phenyl tetrazolium chloride, resazurine or other chromogenic substrates.

Suitable permeable membranes for use in the present invention include, without limitation, nitrocellulose, cellulose, nylon, polyvinylidene fluoride (PVDF) or cellophane.

Suitable permeable membranes for use in the present invention include, without limitation, nitrocellulose, cellulose, nylon, polyvinylidene fluoride (PVDF) or cellophane.

### BRIEF DESCRIPTION OF THE DRAWINGS

The drawings included herewith are incorporated in and form a part of this specification. The drawings illustrate embodiments of the present disclosure and, together with the description, serve to explain the principles of the disclosure. It should be understood that the drawings referred to in this description are not drawn to scale unless specifically noted as such.

FIG. 1 shows (A) a permeable membrane without immobilized antibodies; and (B) a permeable membrane with immobilized antibodies and blocking molecules.

FIG. 2 shows (A) a permeable membrane with immobilized antibodies on the surface of nutrient agar with colonies/microcolonies; and (B) a permeable membrane with slightly visible or invisible (in case of microcolonies) patterns of colonies/microcolonies before washing.

FIG. 3 shows (A) a washing device filled with a washing solution and having a permeable membrane mounted inside with a magnetic stirrer; (B) a top view of the washing device; and (C) a permeable membrane with invisible immobilized antibodies and invisible patterns of colonies/microcolonies.

FIG. 4 shows (A) a permeable membrane with invisible colonies/microcolonies on the surface of gel/agar filled with a chromogenic or fluorogenic substrate or their mixtures which becomes visible after a short incubation; and (B) a permeable membrane with visible colonies/microcolonies after a short incubation.

FIG. 5 shows a permeable membrane which is ready to be placed on a drop of conjugate (antibodies+enzyme) and incubated for a few minutes for specific binding of the conjugate to target cells/patterns.

FIG. 6 shows (A) a permeable membrane with immobilized antibodies and specifically attached cells/patterns with specifically attached conjugate ready for staining; and (B) colored patterns of target colonies/microcolonies.

FIG. 7 is a general scheme of invention methods with five primary detection, identification and/or enumeration steps (1-5) and five confirmation steps (6-10).

### DETAILED DESCRIPTION OF THE INVENTION

The present invention provides an apparatus and methods that overcome the limitations of the prior art and that they are quick, simple, inexpensive and accurate for selectively detecting and identifying live microorganisms. The apparatus and methods of the invention are several times faster than commonly used methods. In addition, the invention is much less costly than very expensive and sophisticated equipment like flow cytometers, mass-spectrometers, FTIR spectroscopy, and PCR. The present invention also has the distinct advantage in that it is applicable to living cells.

The present invention is based on the ability of antigens of live cells, i.e., microorganisms, to bind to antibodies which are immobilized on the surface of a permeable membrane that allows substances, but not microorganisms, to pass through the membrane; on the ability of antibodies to be immobilized directly or through an additional binding agent on to the surface of a permeable membrane; on the ability of microorganisms to grow on solid or in liquid nutrient media or on the surface of a filtration membrane placed on nutrient media and to form microcolonies thereon; and on the ability of water-soluble chromogenic or fluorogenic substrates to pass through a permeable membrane and to react with live microorganisms on the surface of the permeable membrane followed by chromogenic or fluorescent staining of the microorganisms.

As used herein, the terms “cells” and “microorganisms” are interchangeable.

Microorganisms that are placed on a permeable membrane are able to form microcolonies in several hours, for example, 5-6 hours for E. coli, Bacillus spp., Staphylococcus and other bacteria; 12-17 hours for Listeria monocytogenes, Streptococcus B. and others; 15-20 hours for yeasts. This amount of time is only about one-fourth to about one-third the time needed to identify microorganisms compared to the growth times needed for identification of microorganisms using conventional methods. Thus, microcolonies grown on the permeable membranes of the present invention can be identified much sooner than what can be achieved using methods of the prior art.

The permeable membrane used in the present invention, such as a nitrocellulose membrane, which usually is used for filtration purposes, is able to bind proteins on its surface because of the strong interaction of nitrite (NO₂) groups contained in the nitrocellulose with amine (NH₂) groups contained in antibodies. The nitrocellulose membrane thus is able to immobilize antibodies specific to target microorganisms in spaces of the membrane, i.e., antibodies are immobilized in the permeable membrane. Spaces not filled with antibodies are filled with a blocking buffer so that all spaces of the permeable membrane are filled.

Permeable membranes made from other materials may also be used in the invention, such as nylon, PVDF, cellulose or cellophane.

In the methods of the invention, a blocked permeable membrane, such as a nitrocellulose membrane, with immobilized antibodies thereon, is mounted for about 0.5 to 1 minute on the surface of a container filled with nutrient medium, such as agar, on a filtration membrane, or in a liquid medium, each of which contains live cells which contain antigens that react with the immobilized antibodies that target specific microorganisms/microcolonies. Non-target cells also have the ability to stick to the permeable membranes of the invention. Therefore, after mounting of the membrane on the live cells, the membrane needs to be washed thoroughly for about 1 to 3 minutes in washing buffer, such as PBS with 0.1% Tween-20 in a washing device.

After thorough washing of the permeable membrane, the permeable membrane has invisible replicas of microcolonies thereon. The permeable membrane then is placed on a plate filled with agar having dissolved therein one or more chromogenic substrates and/or fluorogenic substrates for rapid, i.e., about 5 to 10 minutes, coloration and/or fluorescence and visualization. Target microorganisms of the present invention bind to the permeable membrane in much larger numbers than non-target microorganisms. All cells are remain in a live and active condition. After about 5 to 10 minutes of incubation on agar with a chromogenic substrates such as MTT, for example, the cells stain an intense dark violet color. Replicas of target cells have much more coloration because the number of bound target cells exceed the number of non-target cells by 5 to 50-fold.

Most non-target replicas are completely washed out from the permeable membrane. However, some non-target replicas do not wash out completely and reveal a weak coloration. Thus, target and non-target cells can be simply and rapidly differentiated from each other and then enumerated.

Microbiological analysis typically requires different levels of reliability of results. For example, epidemiology of deadly infections, bio-terrorism, antibiotic-resistant bacteria or yeasts in immune-suppressed patients requires a very high level of reliability. In contrast, scientific research, some environmental analyses, food microbiology of non-infective microorganisms, for example, requires a lower level of reliability. The present invention allows for raising the reliability of results significantly by providing several additional steps based on the use of enzyme-antibody conjugates. A permeable membrane having stained replicas thereon can be treated with a specific enzyme-antibody conjugate, such as a horseradish peroxidase (HRP)-antibody. A permeable membrane of the present invention can be placed “face” down on a drop containing a dilution of a conjugate in PBS (pH 7.2-7.4). The permeable membrane then is incubated with the conjugate for about 3 to 5 minutes at room temperature. The conjugate binds to all target cells and fills the body of the permeable membrane. Non-bound conjugate molecules then are thoroughly washed out, for about 3 minutes, to prevent non-specific staining. After washing, the permeable membrane is placed on a drop containing a staining solution such as DAB (3 mg/ml in Tris Saline buffer pH 7.6), 3% H₂O₂ and 1.2 weight % of NiCl₂. A color reaction rapidly develops in about 0.5 to 3 minutes on the surface of the permeable membrane in places where microcolonies of target microorganisms are located. The colored spots appear as grey-brown round dots. Violet spots of non-target microorganisms usually disappear but may be visible. The color, intensity and size of the spots of the target spots versus non-target spots differ dramatically.

The present invention provides for the rapid detection and identification of live cells in liquid samples. A blocked permeable membrane with immobilized antibodies thereon is placed on or in a liquid sample that may contain target microorganisms. Target cells bind to the immobilized antibodies. Non-target cells then are washed out from the permeable membrane. Bound target cells then are grown on the permeable membrane to form microcolonies. The time for such growth typically is about 5 to 7 hours, which is one-fourth to one-third less time than usual. The permeable membrane with microcolonies grown thereon then is placed on a staining medium, such as MTT, other chromogens or a combination of chromogens, for visualization and enumeration of the microcolonies.

Because there is non-specific binding of non-target cells, to improve reliability antibodies can be immobilized on a portion of the permeable membrane or parts of the membrane to form shapes of letters or signs. The portion of the membrane without immobilized antibodies serves as a control as the number of microcolonies appearing in this portion shows the number or percentage of non-specific cell binding. Thus, the difference between the number of microcolonies in non-immobilized areas and immobilized areas provides an accurate number of target microorganisms.

The present invention allows for the immobilization of two or more different antibodies on a permeable membrane to bind to different target cells in a sample, which can be differentiated by the size of the microcolony/replica or by the color if a mixture of different chromogenic substrates with different colors are used. For example, E. coli forms much larger microcolonies than Salmonella species in the same amount of time. In the case where both microorganisms are present in a sample, they can be differentiated by the size of their respective colored spots on the permeable membrane. Another option for identification of two or more species is the use of a mixture of different chromogenic substrates giving different colored spots if mixed in staining media. For example, E. coli cells react with MTT by producing a violet color but does not produce any color with INT. However, Micrococcus luteus stains with MTT, producing a violet color and stains even more intensively with INT, producing a red color. A mixture of these two substrates thus allows for a simple and rapid differentiation of different microcolonies by color.

Both monoclonal, polyclonal or a combination of the two antibodies can be used for immobilization on permeable membranes and in enzyme-antibody conjugates. Lectins or other natural agglutinins may also be used in place of antibodies.

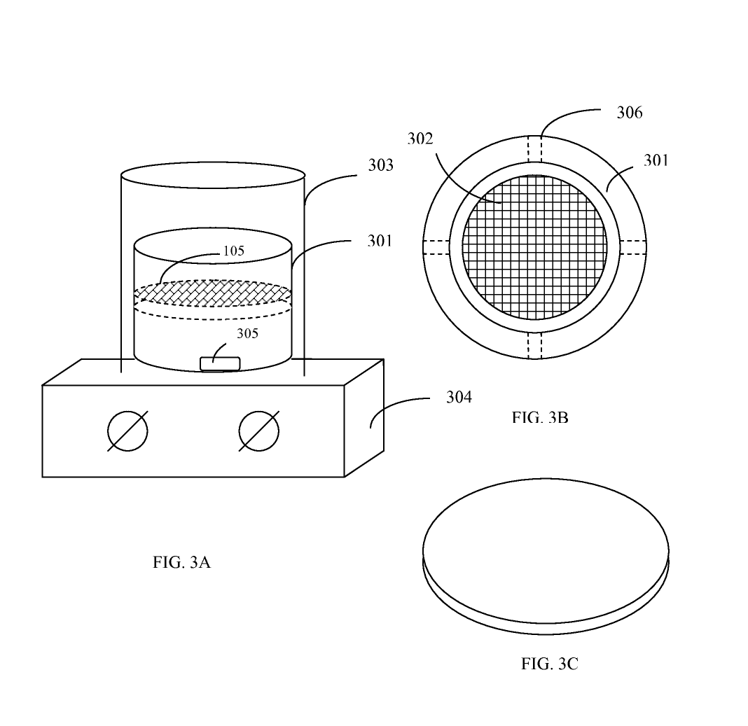

Washing is very important step in the identification procedure of the present invention. Washing must be done thoroughly and precisely for results to be reproducible. Washing is performed during antibody immobilization, during washing out of non-target cells and for washing out non-bound conjugates. The composition of the washing liquid can be the same for all of these steps: PBS pH 7.4 and 0.1% of Tween 20. FIG. 3A, B A shows a simple and effective washing device in a glass beaker having a magnet on the bottom of the beaker and a regular magnetic stirrer. A plastic or metal grid allows for constant rotation of the wash liquid to effectively wash the permeable membrane. Holes in the upper and lower parts of the washing device help to provide rapid liquid exchange inside the glass. The rotating liquid squeezes the membrane against the grid to provide precise washing as the stream of the liquid washes the surface of the membrane with the same speed and force. Washing time typically is about 1 to 3 minutes.

Chromogenic substrates best suitable for the present invention are those that produce color rapidly and intensively after a membrane with cells thereon is placed on a staining medium, such as, for example, tetrazolium salts, resazurine or other chromogenic substrates. In addition, fluorogenic substrates producing fluorescence substances with enzymes of live cells attached to a permeable membrane as a result of an antibody-antigen interaction can be used as well. All substrates suitable in the present invention allow for the free and normal growth of microcolonies on permeable membranes.

The present invention allows for the identification of many different kinds of microcolony-forming microorganisms, such as bacteria, yeasts, fungi, spores or eukaryotic cells. In addition, antigens, such as cells, viruses or proteins, which can bind to a permeable membrane in large concentrations can be identified even without preliminary growth and are able to be visible on a colored permeable membrane.

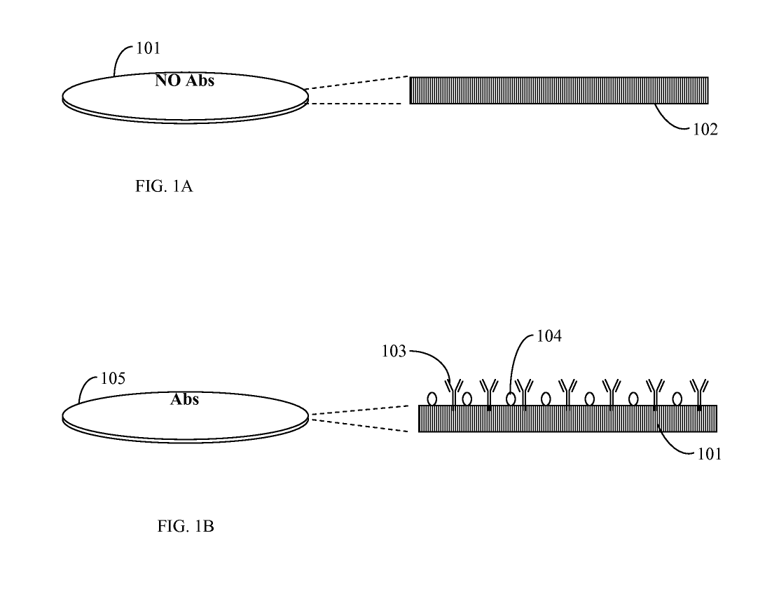

Referring to FIG. 1A, a permeable membrane 101 without immobilized antibodies (“NO Ab”) is shown. The permeable membrane can be made from any material 102 with suitable filtration and antibody binding properties, such as nitrocellulose, cellulose acetate, polycarbonate, PVTF or cellulosic filtration membrane, BioNanoPore membrane, regenerated cellulose or other membranes that are permeable for water and substances but not permeable for cells or target cells. A permeable membrane 101 can be any suitable color for best viewing the bound or target microorganisms that are stained, for example, a white membrane for dark stained microorganisms, a dark colored membrane for fluorescent stained microorganisms, and non-fluorescent transparent or translucent.

Referring to FIG. 1B, a permeable membrane 105 with immobilized antibodies (“Ab”) is shown. Atop the material having filtration properties 102 are immobilized antibodies 103 (e.g., monoclonal antibodies, polyclonal antibodies or non-specific lectins) and a blocking agent 104 (e.g., bovine serum albumin or milk blocking buffer). Immobilization of antibodies can be performed by different methods known in the art. One membrane can have one, two, three or more different antibodies immobilized thereon in order to identify one, two, three or more targets.

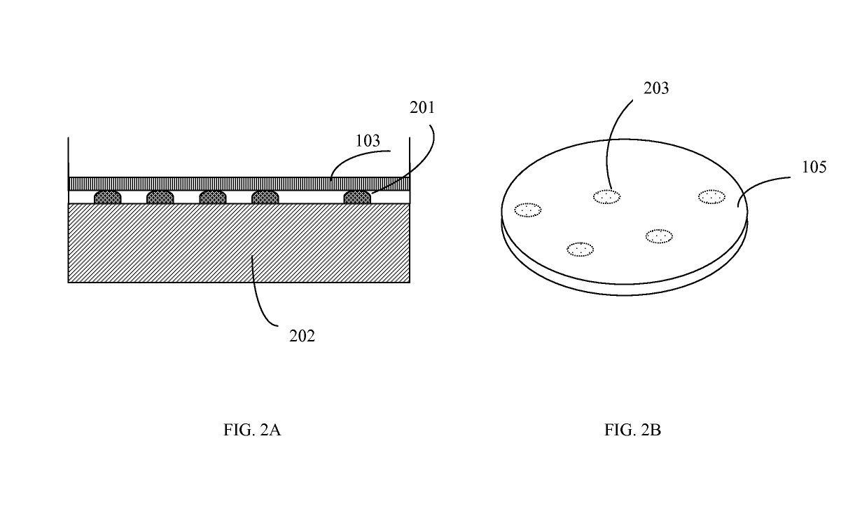

Referring to FIG. 2A, a permeable membrane 103 with immobilized antibodies thereon is placed on the surface of solid nutrient agar 201 with growing microcolonies is shown. Microcolonies can be in different stages of growth but more earlier stages such as about one-fourth to one-third of regular growth time are preferable in order to perform faster analysis. Usually, microcolonies are invisible because of their small size and absence of color. Different nutrient agars can be used as long as they are able to maintain faster cell growth.

Referring to FIG. 2B, a permeable membrane 105 with immobilized antibodies and replicas of microcolonies 203 are shown. Colorless microcolonies are practically invisible on a background of a white filtration membrane. A good material for making replicas is nitrocellulose membrane because cells bind to dry or wet nitrocellulose membranes rapidly and more or less strongly, but other materials like nylon or PVDF can be used as well. Usually, the membrane is placed on the surface of agar having microcolonies thereon for about 0.5 to 1 minute in order to bind all of the microcolonies to the surface of the membrane by an antibody-antigen interaction, i.e., an NO₂—NH₂ interaction, and by capillary forces inside the dry membrane.

Referring to FIG. 3A, a washing device for gentle and precisely reproducible washing of non-target cells is shown. It consists of a plastic or metal cylinder with a ledge 301 to hold a grid with big holes 302 therein made from plastic or metal. A cylinder also has big size holes 306 for faster circulation of the washing solution inside the washing volume. The construction of the washing device ensures that the membrane during the washing process is strongly pressed against the grid by hydrodynamic forces but does not dangle inside the washing volume, which would cause unpredictable washing conditions. The washing device installed in the glass beaker 303 is placed on a magnetic stirrer 304 equipped by a magnet bar regulator 305 for speed of rotation. Thus, speed of washing solution rotation can be precisely maintained to ensure reproducibility of results. Usually, the speed of rotation is set in the middle position. The washing solution can be, for example, PBS pH 7.4 with 0.1% of Tween 20.

Referring to FIG. 3B, a top view of a washing device is shown. It is a cylindrical structure produced from metal or plastic consisting of a wall 301, a grid 302 and several large holes 306 for rapid liquid circulation and better washing conditions.

Referring to FIG. 3C, a permeable membrane 105 after 2 minutes of washing is shown. It looks like a clean membrane without of any signs of replicas. Nevertheless thin (e.g., a one cell thickness layer) invisible spots consisting of tens of thousands to hundreds of thousands of live cells are present on the membrane.

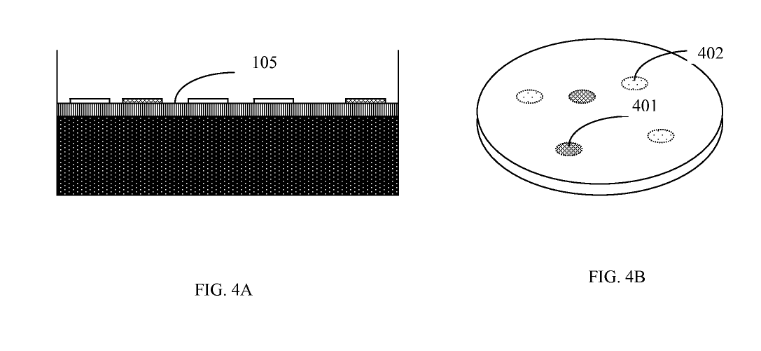

Referring to FIG. 4A, a permeable membrane with initially invisible replicas on the surface of a gel filled by chromogenic or fluorogenic substrates or their mixtures which becomes visible after short incubation is shown. Incubation of the membrane with invisible replicas takes about several minutes. For example, MTT staining of live cells through a reaction with dehydrogenases produces an intense dark-violet color and requires only about 5 to 10 minutes of staining at room temperature. The use of other tetrazolium salts or other chromogenic substrates may require other incubation times. Chromogenic substrates are mainly water soluble. The membrane 105 is placed on the medium by the side free of replicas and molecules of chromogen moves to the invisible replicas by diffusion through the permeable membrane.

Referring to FIG. 4B, a permeable membrane with colonies/microcolonies/replicas visible after a short incubation is shown. Replicas of a target microorganism 401 look much more intensively stained in comparison with non-target replicas 402 because the number of cells in the target replicas is ten to one-hundreds times higher due to the antigen-antibody interaction.

In addition to the intensity of staining of the replicas, other features of the replicas can be employed for identification in the present invention. For example, the size of a stained spot depends on the speed of growth which can help to differentiate target versus non-target replicas. In addition, the use of a mixture of chromogenic substrates or a mixture of chromogenic substrates and other substances with a specific response can be used as well.

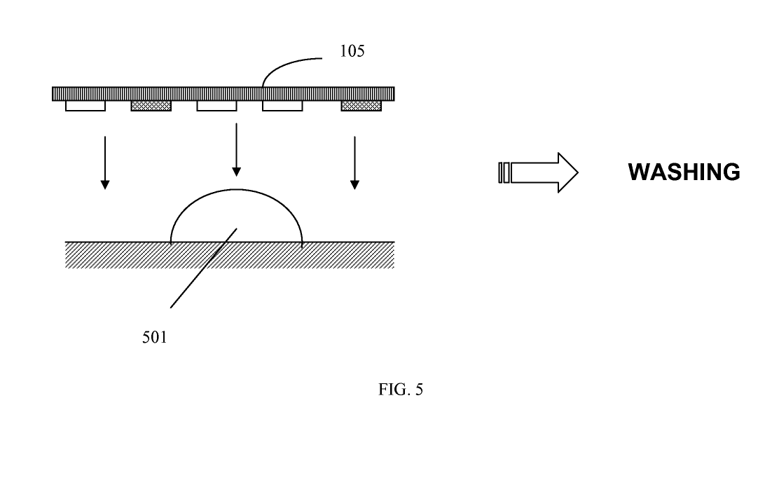

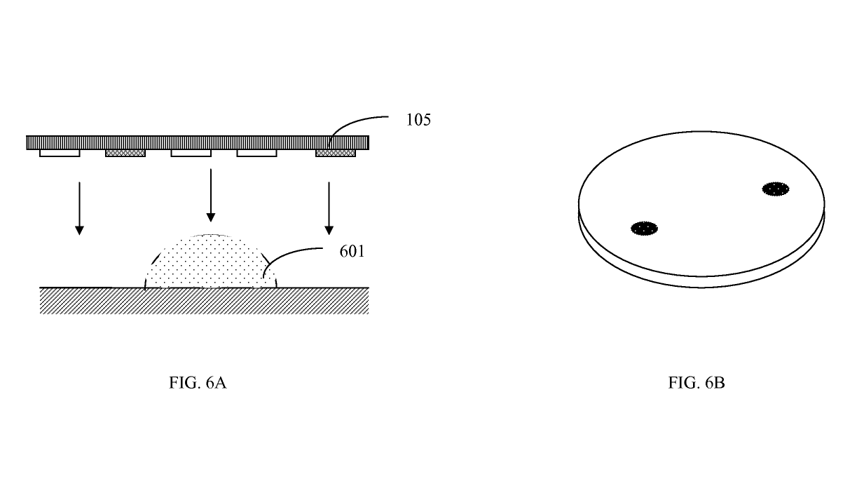

If there is little difference in the color intensity of target versus non-target replicas, additional analytical reactions can be performed. Thus, replicas containing target cells can be analyzed by enzyme immunoanalysis on permeable membranes. For instance, a permeable membrane with stained replicas can be placed ‘face down’ on a drop containing a conjugate (antibodies bound to an enzyme, such as HRP or others) 501 is shown in FIG. 5. A permeable membrane 105 that is ready to be placed on a drop of conjugate (antibodies+enzyme) and incubated for a few minutes to specifically bind the conjugate to target cells/patterns is shown on the FIG. 5. Non-reacted conjugates thoroughly wash out with a help of the washing device shown in FIG. 3A, B. Only target cells bind to molecules of conjugate associated with the enzyme. The most widely used enzyme in such conjugates is horseradish peroxidase (HRP). It can be uncovered in a reaction with diaminobenzidine (DAB), NiCl₂ and H₂O₂. A washed membrane with stained replicas placed “face down” on a drop containing DAB, NiCl₂ and H₂O₂ 601 is shown in FIG. 6A. Replicas of target microorganisms obtain a strong dark-grey color as a result of a reaction between a staining mixture and HRP associated with antibodies as shown in FIG. 6B. Other replicas retain their color or disappear in cases where MTT is used. Non-specific peroxidase activity, which kills cells due to crystallizing cellular organelles is lowered by the use of MTT.

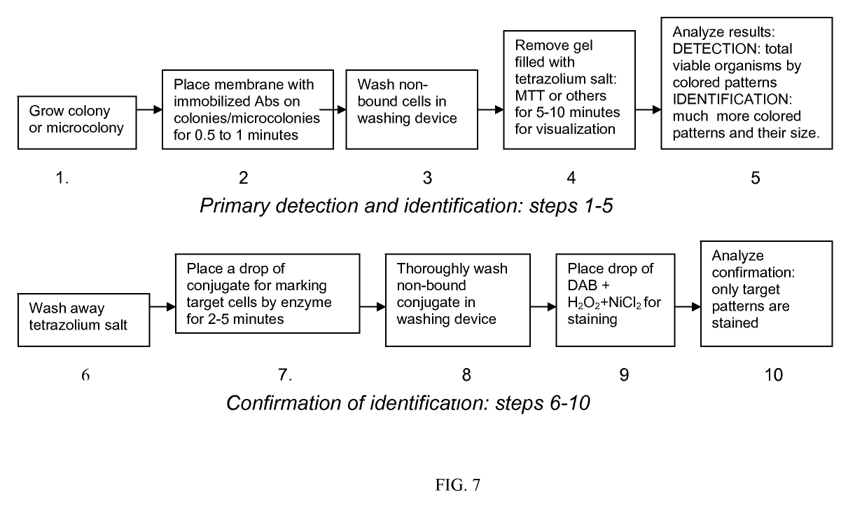

The general scheme of the present invention is shown in FIG. 7. Only the first five steps (primary detection and identification) need be performed in the case where stained spots show a big difference in intensity of coloration. Confirmation of the result or additional staining of replicas with the use of enzyme conjugates and a staining solution is shown in the last five steps of analysis.

### EXAMPLES

The present invention is more particularly described in the following non-limiting examples, which are intended to be illustrative only, as numerous modifications and variations therein will be apparent to those skilled in the art.

### Example 1

### Rapid Detection and Identification of One Target

A sample containing an unknown mixture of species including a possible target microorganism such as E. coli O157 H:7 was poured on a Petri plate filled with nutrient agar. A plate with microorganisms was incubated at a temperature optimal for the target microorganism for 5 to 6 hours or more. A transfer permeable membrane containing immobilized antibodies against E. coli O157 H:7 and blocked in 1% of bovine serum albumin overnight was mounted on the surface of nutrient media and incubated for approximately 0.5 to 1 minute. This time was sufficient for an antigen-antibody reaction to occur and reliable attaching of one layer of cells to immobilized antibodies. Next, the transfer membrane was washed in washing solution (PBS pH 7.4 with 0.1% Tween 20) in a washing device at medium speed of rotation for 2 minutes. The transfer membrane was placed on a staining plate containing agar with dissolved 3-(4,5-Dimethylthiazol-2-yl)-2,5-diphenyltetrazolium bromide (MTT) which penetrated the membrane and colored cells to a strong deep violet color in a matter of minutes. The stained patterns of microcolonies (replicas) were well visible on the white background of the transferring membrane. These patterns could be found and enumerated. In case confirmation of analysis was necessary, the next five steps of identification with conjugate and staining mixture could be performed with this membrane.

### Example 2

### Rapid Detection and Identification of Two Targets: E. Coli O157 and Salmonella

This procedure differed from the previous procedure by using a permeable nitrocellulose membrane with two immobilized antibodies for two different targets. Thus, two species left patterns on the transfer membrane. Differentiation of the two species (E. coli O157 and Salmonella) were performed by the use of a mixture of two antibodies immobilized on the surface of a permeable membrane and blocked after immobilization in order to prevent non-specific binding. Thus, a sample containing a mixture of species presumably including E. coli O157 and/or Salmonella was placed on a solid nutrient TSA medium in a Petri plate and grown for 7 to 8 hours at 37° C. Then, the membrane with the two antibodies was placed on the surface for 0.5 to 1 minute, washed in a washing solution for 2 minutes and placed on 1% agar filled with MTT (1 mg/ml). Patterns of E. coli O157 replicas obtained a well visible violet color with a size around 1.5 mm in diameter. Patterns of Salmonella had the same color but a size of only 0.5 mm. Thus, two targets on one membrane could be differentiated simply by the size of equally colored spots.

### Example 3

### Rapid Detection of All Microcolonies

In this procedure, a transfer permeable membrane contained no antibodies. All microcolonies adhered to the surface of the transfer membrane by non-covalent hydrophobic interaction but not by antibody-antigen binding. The washing out step was omitted in order to retain all replicas on the surface of the transfer permeable membrane. Next, the transfer membrane was moved to a staining plate containing MTT or another chromogen without washing. All replicas obtained a very intense dark violet color without washing and could be enumerated at a very early stage of growth: after only 4 to 6 hours. This was very important for early and rapid analysis of all live cells in a sample. The chromogenic substrate could be immobilized on the permeable membrane instead of being dissolved in the agar. This could simplify analysis because it would cut out two steps: washing in washing solution and transfer onto a staining plate (agar with a chromogenic substrate such as MTT). In this case, the transfer permeable membrane needed to be placed with the immobilized chromogenic substrate on the surface containing invisible microcolonies for 3 to 5 minutes. Then the membrane was removed and dark spots/replicas of microcolonies corresponding to live cells in the sample were enumerated.

### Example 4

### Identification After Antibody-Antigen Binding, Growth and Staining

Antibodies immobilized on a permeable membrane were used as a surface for the growth of specifically attached cells and then were stained. This procedure was useful for analyzing a liquid sample presumably containing target microorganisms. Thus, a sample containing E. coli O157 (target) and other species were placed on the surface of a permeable membrane with antibodies immobilized only in the center part of the membrane for 1 to 2 minutes. This time was enough for many target cells to attach to the part of the membrane with antibodies. Then, the membrane was submerged in the washing solution (PBS with 0.1% Tween 20, pH 7.4) in the washing device and washed for 3 minutes. During washing, practically all non-target cells washed out from blocked areas blocked with 1% Bovine Serum Albumin or another blocker, while cells E. coli O157 remained immobilized by antibodies on parts of the membrane. The washed membrane was moved to the nutrient TSA agar surface with cells and incubated for 4 to 5 hours at 37.5° C. Each live cell produced a microcolony on the surface of the membrane containing hundreds or thousands of individual cells. Then, the membrane was placed on agar filled with the chromogenic substrate MTT and all small invisible microcolonies obtained an intense dark violet color and became very visible. The area with immobilized antibodies obtained a distinctive color with well visible stained microcolonies. The surrounding area did not have microcolonies or had ten or one-hundred times less stained microcolonies as a result of non-specific binding or mechanical contamination.

The experiments showed that up to thousands or tens of thousands of E. coli O157 cells in an investigated sample could be revealed in a very short time (3-5 hours) by this very simple and reliable method. The area of antibody immobilization could have different shapes: a round spot, a geometric shape or shaped as letters. A specific shape of an immobilized area was especially useful in case two or more targets needed to be identified in a sample: letters EC (or special sign/shape) could belong to an E. coli immobilized area and letters SL (or special sign/shape) could belong to Salmonella in case both of these species needed to be identified.

These procedures were rapid, taking only one-fourth to one-third regular growth time, visualized only live cells after growth, was very simple because it consisted of a few simple steps did not need special sophisticated equipment and was inexpensive cheap because it needed only cost-effective reagents and minimal labor.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

What is claimed is:

1. A method for rapid detection, identification and/or enumeration of one or more live target microorganisms in a sample, comprising:
placing at least one target microorganism on a nutrient medium to grow at least one microcolony of the at least one target microorganism;
placing a permeable membrane on the at least one microcolony, said permeable membrane having at least one type of immobilized antibody thereon specific to an antigen on the at least one target microorganism in the at least one microcolony in order to bind the at least one target microorganism to the at least one antibody to form a replica of the at least one microcolony on the permeable membrane;
washing away any non-bound microorganisms from the permeable membrane; and
staining the at least one microcolony on the permeable membrane to form a stained replica of the at least one microcolony by placing the permeable membrane on a staining plate containing at least one chromogenic substrate, at least one fluorogenic substrate or a combination of the two to obtain colored and/or fluorescent spots of the at least one target microorganism.

2. The method of claim 1, wherein the at least one microorganism is selected from the group consisting of bacteria, yeasts, fungi and eukaryotic cells.

3. The method of claim 1, wherein the nutrient medium is an agar such as tryptic soy agar.

4. The method of claim 1, wherein the plate contains agar having dissolved therein the chromogenic and/or fluorogenic substrates.

5. The method of claim 1, wherein the at least one chromogenic substrate is selected from the group consisting of a tetrazolium dye such as of MTT (3-(4,5-dimethylthiazol-2-yl)-2, 5-diphenyltetrazolium bromide) and INT (2-(p-iodophenyl-3-(p-nitrophenyl)-5-phenyl tetrazolium chloride, resazurine and other chromogenic substrates.

6. The method of claim 1, wherein the at least one type of immobilized antibody is a monoclonal antibody or a polyclonal antibody.

7. The method of claim 1, wherein the permeable membrane is selected from the group consisting of nitrocellulose, cellulose, nylon, polyvinylidene fluoride (PVDF) and cellophane.

8. The method of claim 1, wherein the detection, identification and enumeration is performed on the at least one microcolony after growing the at least one microcolony for about one-fourth to about one-third less time than usual, wherein the usual growth time is between about 24 to about 48 hours for bacteria and about 72 to about 120 hours for fungi.

9. The method of claim 1, wherein the detection, identification and enumeration is performed on the at least one microcolony after growing the at least one microcolony for longer than about one-fourth to about one-third less time than usual, wherein the usual growth time is between about 24 to about 48 hours for bacteria and about 72 to about 120 hours for fungi.

10. The method of claim 1, further comprising:
placing at least one type of antibody-enzyme conjugate on the permeable membrane, said antibody-enzyme conjugate specific to the antigen of the at least one target microorganism of the stained replica;
incubating the permeable membrane for a period of time;
washing any non-bound antibody-enzyme conjugate from the permeable membrane;
placing a staining solution on the permeable membrane to obtain colored spots of the at least one target microorganism in the stained replica, said colored spots differing from colored spots of non-target microorganisms.

11. The method of claim 11, wherein the staining solution is 3,3′-diaminobenzidine (DAB).

12. A method for rapid detection, identification and enumeration of one or more live target microorganisms in a liquid sample, comprising:
placing a permeable membrane having at least one type of immobilized antibody thereon specific to an antigen on the at least one target microorganism; said at least one type of immobilized antibody binding to the antigen on the at least one target microorganism to form at least one microorganism antigen-antibody complex;
washing away any non-bound microorganisms from the permeable membrane;
incubating the at least one microorganism antigen-antibody complex to form at least one microcolony on the permeable membrane; and
staining the at least one microcolony on the permeable membrane by placing the permeable membrane on a plate containing at least one chromogenic substrate, at least one fluorogenic substrate or a combination of the two to obtain colored spots of the at least one target microorganism.

13. The method of claim 12, wherein the at least one type of antibody is immobilized on the entire surface of the permeable membrane, on a portion of the surface of the permeable membrane, or in portions of the permeable membrane to form signs, shapes or letters.

14. The method of claim 12, wherein the at least one microorganism is selected from the group consisting of bacteria, yeasts, fungi and eukaryotic cells.

15. The method of claim 12, wherein the nutrient medium is an agar such as tryptic soy agar.

16. The method of claim 12, wherein the at least one chromogenic substrate is selected from the group consisting of a tetrazolium dye such as of MTT (3-(4,5-dimethylthiazol-2-yl)-2, 5-diphenyltetrazolium bromide) and INT (2-(p-iodophenyl-3-(p-nitrophenyl)-5-phenyl tetrazolium chloride, resazurine and other chromogenic substrates.

17. The method of claim 12, wherein the at least one type of immobilized antibody is a monoclonal antibody or a polyclonal antibody.

18. The method of claim 12, wherein the permeable membrane is selected from the group consisting of nitrocellulose, cellulose, nylon, polyvinylidene fluoride (PVDF) and cellophane.

19. The method of claim 12, wherein the detection, identification and enumeration is performed on the at least one microcolony after growing the at least one microcolony for about one-fourth to about one-third less time than usual, wherein the usual growth time is between about 24 to about 48 hours for bacteria and about 72 to about 120 hours for fungi.

20. The method of claim 12, wherein the detection, identification and enumeration is performed on the at least one microcolony after growing the at least one microcolony for longer than about one-fourth to about one-third less time than usual, wherein the usual growth time is between about 24 to about 48 hours for bacteria and about 72 to about 120 hours for fungi.

21. The method of claim 12, further comprising:
after staining the at least one microcolony on the permeable membrane placing at least one type of antibody-enzyme conjugate on the permeable membrane, said antibody-enzyme conjugate specific to the antigen of the at least one stained target microorganism;
incubating the permeable membrane;
washing any non-bound antibody-enzyme conjugate from the permeable membrane;
placing a staining solution on the permeable membrane to obtain colored spots of the at least one stained target microorganism, said colored spots differing from colored spots of non-target microorganisms.

22. The method of claim 21, wherein the staining solution is 3,3′-diaminobenzidine (DAB).

23. A method for rapid detection, identification and/or enumeration of one or more live target microorganisms, comprising:
placing at least one target microorganism on a nutrient medium to grow at least one microcolony of the at least one target microorganism;
placing a permeable membrane on the at least one microcolony to form a replica of the at least one microcolony on the permeable membrane;
staining the at least one microcolony on the permeable membrane to form a stained replica of the at least one microcolony by using at least one chromogenic substrate, at least one fluorogenic substrate or a combination of the two to obtain colored and/or fluorescent spots of the at least one target microorganism.

24. The method of claim 23, wherein the permeable membrane is placed on a staining plate containing agar having the at least one chromogenic substrate, the at least one fluorogenic substrate or the combination of the two dissolved therein.

25. The method of claim 23, wherein the at least one chromogenic substrate, the at least one fluorogenic substrate or the combination of the two are immobilized on the permeable membrane.

26. A device for rapid detection, identification and enumeration of one or more live target microorganisms in a sample, comprising:
a container having therein nutrient medium to grow at least one microcolony of the at least one live target microorganism;
a permeable membrane that contacts the at least one microcolony grown on the nutrient medium, said permeable membrane having at least one type of immobilized antibody thereon specific to an antigen on the at least one target microorganism in the at least one microcolony in order to bind the at least one target microorganism to the at least one antibody to form a replica of the at least one microcolony on the permeable membrane; and
a plate containing at least one chromogenic substrate, at least one fluorogenic substrate or a combination of the two to obtain colored and/or fluorescent spots of the at least one target microorganism.

27. The device of claim 26, wherein the at least one type of antibody is immobilized on the entire surface of the permeable membrane, on a portion of the surface of the permeable membrane, or in portions of the permeable membrane to form signs, shapes or letters.

28. The device of claim 26, wherein the at least one microorganism is selected from the group consisting of bacteria, yeasts, fungi and eukaryotic cells.

29. The device of claim 26, wherein the nutrient medium is an agar such as tryptic soy agar.

30. The device of claim 26, wherein the plate contains agar having dissolved therein the chromogenic and/or fluorogenic substrates.

31. The device of claim 26, wherein the at least one chromogenic substrate is selected from the group consisting of a tetrazolium dye such as of MTT (3-(4,5-dimethylthiazol-2-yl)-2, 5-diphenyltetrazolium bromide) and INT (2-(p-iodophenyl-3-(p-nitrophenyl)-5-phenyl tetrazolium chloride, resazurine and other chromogenic substrates.

32. The device of claim 26, wherein the at least one type of immobilized antibody is a monoclonal antibody or a polyclonal antibody.

33. The device of claim 26, wherein the permeable membrane is selected from the group consisting of nitrocellulose, cellulose, nylon, polyvinylidene fluoride (PVDF) and cellophane.

34. The device of claim 26, wherein the detection, identification and enumeration is performed on the at least one microcolony after growing the at least one microcolony for about one-fourth to about one-third less time than usual, wherein the usual growth time is between about 24 to about 48 hours for bacteria and about 72 to about 120 hours for fungi.

35. The device of claim 26, wherein the detection, identification and enumeration is performed on the at least one microcolony after growing the at least one microcolony for longer than about one-fourth to about one-third less time than usual, wherein the usual growth time is between about 24 to about 48 hours for bacteria and about 72 to about 120 hours for fungi.

36. A medical device for rapid detection, identification and enumeration of one or more live target microorganisms in a sample, comprising:
a container of nutrient media to grow at least one microcolony of the at least one live target microorganism;
a permeable membrane that contacts the at least one microcolony grown on the nutrient medium, said permeable membrane having at least one type of immobilized antibody thereon specific to an antigen on the at least one target microorganism in the at least one microcolony in order to bind the at least one target microorganism to the at least one antibody to form a replica of the at least one microcolony on the permeable membrane; and
a plate containing at least one chromogenic substrate, at least one fluorogenic substrate or a combination of the two to obtain colored and/or fluorescent spots of the at least one target microorganism.

37. The method of claim 36, wherein the at least one microorganism is selected from the group consisting of bacteria, yeasts, fungi or eukaryotic cells.

38. A test kit system for rapid detection, identification and enumeration of one or more live target microorganisms in a sample, comprising:
a container having therein nutrient medium to grow at least one microcolony of the at least one live target microorganism;
a permeable membrane that contacts the at least one microcolony grown on the nutrient medium, said permeable membrane having at least one type of immobilized antibody thereon specific to an antigen on the at least one target microorganism in the at least one microcolony in order to bind the at least one target microorganism to the at least one antibody to form a replica of the at least one microcolony on the permeable membrane;
a staining plate containing agar and one or more chromogenic and/or fluorogenic substrates to obtain colored and/or fluorescent spots of the at least one target microorganism.
